(19) United States
(12) Patent Application Publication
(10) Pub. No.: US 2022/0022797 A1
(43) Pub. Date: Jan. 27, 2022

(54) EXAMINATION SUPPORT METHOD AND EXAMINATION SUPPORT SYSTEM

(71) Applicant: Toray Industries, Inc., Tokyo-to (JP)

(72) Inventors: Shigeo Fujimori, Tokyo (JP); Ichiro Itagaki, Tokyo (JP)

(21) Appl. No.: 17/297,022

(22) PCT Filed: Nov. 29, 2019
(86) PCT No.: PCT/JP2019/046717

§ 371 Date: May 26, 2021

(51) Int. Cl. A61B 5/332; A61B 5/318; A61B 5/282; A61B 5/00; A61B 5/339

(52) U.S. Cl. CPC A61B 5/332; A61B 5/318; A61B 5/282; A61B 2562/04; A61B 5/339; A61B 5/0006; A61B 5/6805

(57) ABSTRACT

An examination support method that supports an electrocardiogram examination of a subject using a garment on which medical equipment including cardiography equipment and a plurality of electrodes are loaded, the examination support method including: a first apparatus selecting a garment to be put on the subject from the garments of a plurality of sizes on a basis of size information on the subject; and a second apparatus connecting by wire or wirelessly with the medical equipment loaded on the garment that the subject has put on and reading out measurement data measured during a period in which the subject puts on the garment.

### TECHNICAL FIELD

This disclosure relates to an examination support method and an examination support system that supports an electrocardiogram examination utilizing a garment that measures biological signals of a subject.

### BACKGROUND

An electrocardiogram is widely used in diagnosis of heart diseases such as angina (cardiac infarction) and abnormal cardiac rhythm. Heart diseases do not always appear in electrocardiograms as abnormalities. An abnormality appears in an electrocardiogram upon occurrence of an attack, and thus, an electrocardiogram when an attack does not occur cannot be distinguished from a normal electrocardiogram. Thus, an examination of taking an electrocardiogram for a long period under stress of exercise and in daily life is performed. Typically, a Holter electrocardiogram examination for 24 hours is widely performed as an electrocardiogram examination for a long period. While this electrocardiogram examination is common, missing of a heart disease is pointed out also in the electrocardiogram examination for 24 hours, and a long-term electrocardiogram examination for one week or longer is desired.

As a method of recording an electrocardiogram comfortably and easily under a daily living environment, there is a method of loading electrodes and measurement equipment on a garment for a subject and measuring biological signals of the subject. According to that method, the subject can be subjected to measurement for a long period equal to or longer than one week in a comfortable environment. Meanwhile, it is important to properly fit the subject. For example, when the size is not correct, electrodes are not fixed at appropriate positions with an appropriate pressure when the subject puts on the garment, which results in failing to obtain stable information with less noise. Further, in the Holter electrocardiogram examination in related art, a subject needs to visit the hospital a number of times (for example, equal to or more than four times) from when a doctor makes a diagnosis until when the subject receives feedback of an examination result, which is heavy load on the subject. Thus, an examination method with less load on the subject is desired.

Meanwhile, as a method of supporting an examination of a patient in a daily living environment, there is a support system of medical equipment disclosed in Japanese Patent Laid-Open No. 2002-109073. In that system, an information provision site determines medical equipment to be provided to a patient at home on the basis of information in accordance with a result of counseling with the patient at home and transmits information regarding the determined medical equipment to a server of a medical equipment support company. The medical equipment support company specifies medical equipment to be provided to the patient at home on the basis of the information received at the server and delivers the specified medical equipment to the patient at home. In this manner, that system provides support of the medical equipment to the patient at home. However, there is no disclosure in Japanese Patent Laid-Open No. 2002-109073 regarding how medical equipment suitable for the patient at home is specifically determined. Particularly, there is no disclosure regarding how an electrocardiogram examination of the patient at home is supported.

It could therefore be helpful to provide an examination support method and an examination support system that supports an electrocardiogram examination utilizing a system that measures biological signals of a subject.

### SUMMARY

We thus provide:

An examination support method of supporting an electrocardiogram examination of a subject using a garment on which medical equipment including cardiography equipment and a plurality of electrodes are loaded, the examination support method includes a first apparatus selecting a garment to be put on the subject from the garments of a plurality of sizes on a basis of size information on the subject; and a second apparatus connecting by wire or wirelessly with the medical equipment loaded on the garment which the subject has put on and reading out measurement data measured during a period in which the subject puts on the garment.

An examination support system that supports an electrocardiogram measurement of a subject using a garment on which medical equipment including cardiography equipment and a plurality of electrodes are loaded, the examination support system includes an information processing apparatus that comprises: an input device configured to input size information on the subject; and a first communication device configured to transmit order instruction information on the garment on a basis of the size information; an order reception apparatus which comprises: a second communication device configured to receive the order instruction information from the information processing apparatus; and a garment selector configured to select a garment to be put on the subject from the garments of a plurality of sizes in accordance with the order instruction information; and a data processing apparatus that comprises: a reader configured to connect by wire or wirelessly with the medical equipment loaded on the garment which the subject has put on and read out measurement data measured during a period in which the subject puts on the garment.

It is thus possible to provide an examination support method and an examination support system that can efficiently perform a series of procedure regarding an electrocardiogram examination while reducing load on a subject.

### BRIEF DESCRIPTION OF THE DRAWINGS

FIG. 1 is a block diagram illustrating an entire configuration of an examination support system according to an example.

FIG. 2 is a front view illustrating an example of a measurement garment.

FIG. 3 is a rear view of a front body of the measurement garment according to the example.

FIG. 4 is an enlarged view of a size adjustment mechanism of the front body of the measurement garment.

FIG. 5 is a rear view of the measurement garment according to the example.

FIG. 6 is a view illustrating an example of an order input screen.

FIG. 7 is a view illustrating an example of a garment selection screen according to the example.

FIG. 8 is a view illustrating an example of the garment selection screen.

FIG. 9 is a view illustrating an example of a shipment screen according to the example.

FIG. 10 is a view illustrating an example where fastened positions are marked.

FIG. 11 is a view of a subject seen from a diagonally forward right direction when the subject puts on the measurement garment according to the example.

FIG. 12 is a rear view of the subject seen from a diagonally backward left direction when the subject puts on the measurement garment according to the example.

FIG. 13 is a view illustrating an overview of an analysis result according to the example.

FIG. 14 is a view illustrating part of measured waveforms registered in an analysis report according to the example.

FIG. 15 is a view illustrating part of compressed waveforms of the analysis report according to the example.

FIG. 16 is a sequence diagram of the whole operation according to the example.

### REFERENCE SIGNS LIST

1 measurement garment (garment)
10 cardiography equipment (measurement equipment)
20, 21, 22 electrode
30 garment body portion
31 front body
32 rear body
33 shoulder belt
34 torso portion
35 elastic body
36 side tab
37 connector
40 hook and loop fastener B side
41 stitch
100 information processing apparatus
200 order reception apparatus
300 data processing apparatus
400 inventory management apparatus
500 analysis apparatus
600 communication terminal
700 communication network
101, 201, 301 input device
102, 202, 302 display
103, 203, 303 storage
104, 204, 304 communication device
105 order generator
205 garment selector
206 shipment instruction device
305 reader
306 analysis request device
401 warehouse

### DETAILED DESCRIPTION

An outline of an example will be described first. A subject visits a medical institution, and a doctor diagnoses the subject. When the doctor judges that the subject is suspected to have a heart disease such as angina (cardiac infarction) and abnormal cardiac rhythm, the doctor determines the need to perform a Holter electrocardiogram examination (hereinafter, an electrocardiogram examination) and prescribes an electrocardiogram examination. A laboratory technician measures the body size of the subject as information necessary to determine the size of the measurement garment (garment) to be put on and obtains size information on the subject including an under bust size. Health personnel (a doctor, a laboratory technician or other health personnel to whom the doctor provides directions) of the medical institution orders a garment to be put on the subject to a medical equipment sales agency using an information processing apparatus. The health personnel also notifies the size information on the subject upon ordering. The agency determines a garment of a size appropriate for the subject on the basis of the size information on the subject and sends the determined garment of the size to the subject along with an instruction manual. The subject who has received the garment puts on the garment along the instruction manual and measures biological signals (electrocardiogram signals) during a measurement period (for example, one week). After the measurement period, the subject sends back the garment which the subject has put on to the medical equipment sales agency. The medical equipment sales agency reads out measurement data from measurement equipment (cardiography equipment) loaded on the garment and requests analysis of the readout measurement data to an electrocardiographic analysis institution. The electrocardiographic analysis institution analyzes the measurement data and creates an analysis report. The medical institution receives the analysis report from the electrocardiographic analysis institution, and the doctor diagnoses a state of the subject using the analysis report.

We provide an examination support method and an examination support system that efficiently performs a series of procedure regarding an electrocardiogram examination as described above while reducing load on the subject.

An example will be described in detail below with reference to the drawings.

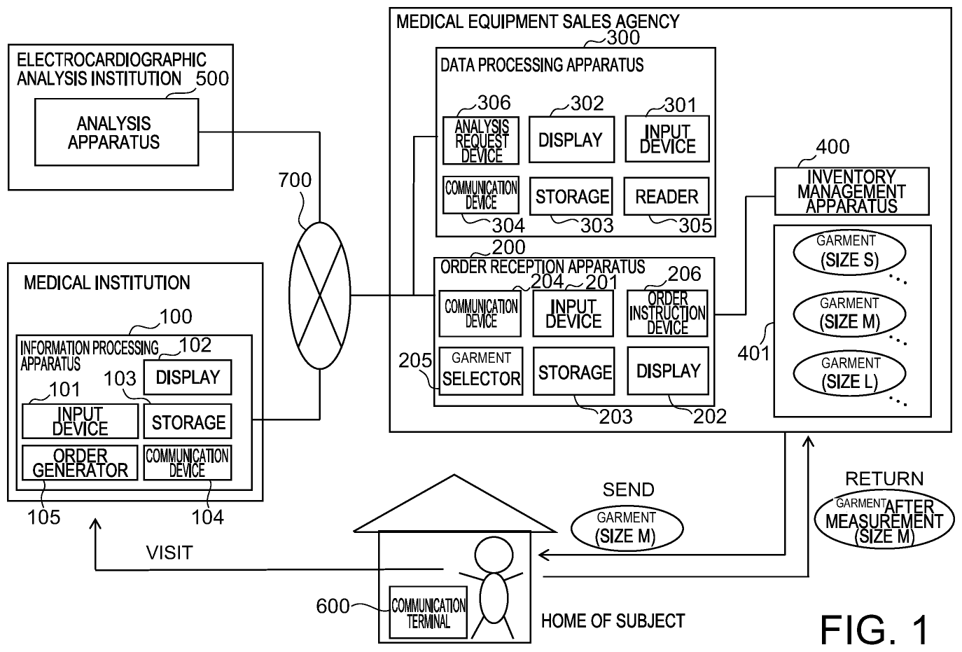

FIG. 1 is a block diagram illustrating an entire configuration of an examination support system according to the example. The examination support system in FIG. 1 includes an information processing apparatus 100 disposed at a medical institution, an order reception apparatus 200, a data processing apparatus 300 and an inventory management apparatus 400 disposed at a medical equipment sales agency, an analysis apparatus 500 disposed at an analysis center, a communication terminal 600 of the subject and a communication network 700.

The medical institution is an institution that provides medical service including an electrocardiogram examination, and the like, to the subject. As an example, the medical institution is a hospital, and the subject is a patient who is to undergo an electrocardiogram examination. A doctor diagnoses the subject, and when the doctor judges that the subject is suspected to have a heart disease such as abnormal cardiac rhythm, the doctor determines to perform an electrocardiogram examination and prescribes the electrocardiogram examination.

The electrocardiogram examination uses a measurement garment (hereinafter, a garment) on which measurement equipment (cardiography equipment) that measures action potential or an action current of cardiac muscle associated with beat of a heart as a biological signal of the subject is loaded. The subject puts on the garment during a measurement period. The measurement period is determined by the doctor and is approximately from one to two weeks as an example. Measurement data recorded by the cardiography equipment is called an electrocardiogram. To order a garment to be put on the subject, a laboratory technician measures the body size of the subject as information necessary to determine the size of the garment. However, the measurement may be performed by the doctor or other health personnel to whom the doctor provides directions. Further, measurement may be performed using a measurement apparatus that automatically measures the body size, or the laboratory technician or the like may manually measure the body size using a measuring tape.

Measurement items of the body size include body height, chest measurement, under bust, shoulder width, sitting height, body weight and the like. The measurement items do not have to include all of these and may include other items. However, it is assumed in the example that the measurement items include at least under bust.

A garment to be used in the example will be described.

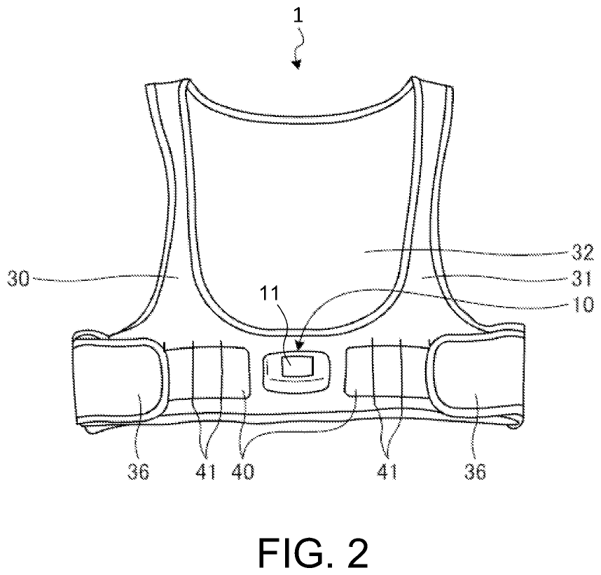

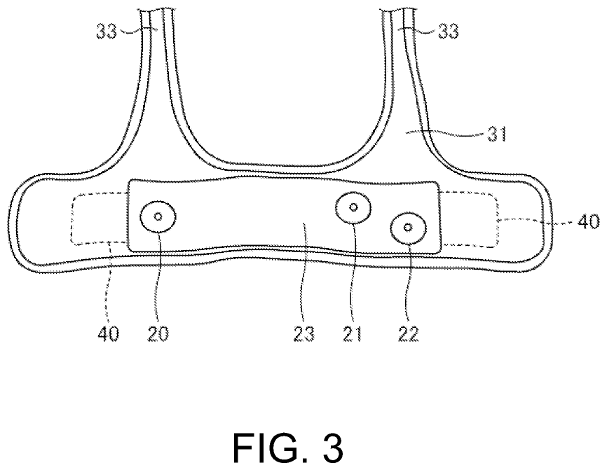

FIG. 2 is a front view of a garment 1. FIG. 3 is a view of a reverse side (side in contact with the skin) of a front body of the garment 1. The garment 1 includes cardiography equipment 10 which is measurement equipment, electrodes 20, 21 and 22 (FIG. 3), and a garment body portion 30. Medical equipment in the example includes the cardiography equipment 10 and the electrodes 20, 21 and 22.

The garment body portion 30 includes a front body 31 and a rear body 32. The front body 31 is connected to the rear body 32 with two shoulder belts (shoulder straps) 33 illustrated in FIG. 3, and the front body 31 is separated from the rear body 32 at both sides (side portions). The cardiography equipment 10 is loaded at the center of a torso portion of the front body 31. On the rear side of the torso portion of the front body 31 on which the cardiography equipment 10 is loaded, electrodes 20, 21 and 22 (FIG. 3) which make contact with the skin of the subject are attached.

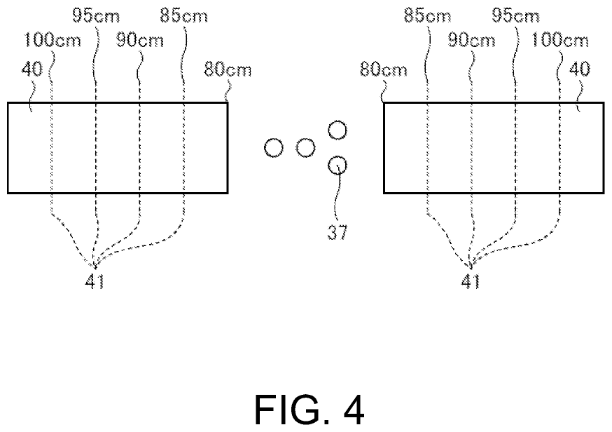

These electrodes 20, 21 and 22 are arranged on the basis of CC5 which is one of induction methods of the Holter electrocardiogram examination. The cardiography equipment 10 and the electrodes 20, 21 and 22 are located around the under bust of the wearer when the wearer puts on the garment. The electrode 20 becomes a positive electrode, the electrode 21 becomes a negative electrode, and the electrode 22 becomes an earth electrode. The electrodes 20, 21 and 22 are constituted with conductive fiber as an example. However, the electrodes 20, 21 and 22 are not limited to conductive fiber, and may be adhesive films including a conductive material. A size and a shape of the electrodes 20, 21 and 22 are not particularly limited as long as the electrodes can detect biological signals. While not illustrated, the respective electrodes 20, 21 and 22 are connected to a connector 37 (see FIG. 4) of the cardiography equipment 10 with a lead line. These wiring portions are covered with an electronic insulating member 23.

The electrodes 20, 21 and 22 may be detachable from the front body 31. Further, the cardiography equipment 10 may be detachable from the garment body portion 30 via the connector 37 (see FIG. 4). By detaching at least the cardiography equipment 10 among the cardiography equipment 10 and the electrodes 20 to 22 from the garment body portion 30, it is possible to wash the garment. As the connector 37, a socket or the like which is typically used in connection of cords can be used as an example. A plurality of metal dot buttons which can fix the cardiography equipment 10 at the garment body portion 30 at the same time may be used.

The cardiography equipment 10 includes a chargeable battery and can record electrocardiogram data (measurement data) for one or two weeks, or more weeks without being additionally charged, in a state where the battery is fully charged. The cardiography equipment 10 includes a storage which can store all data measured during one or two weeks or more weeks. The cardiography equipment 10 includes a communication device which performs communication with apparatuses such as a computer by wire or wirelessly. The communication device has a function of connecting with the apparatus by wire or wirelessly and transmitting the measurement data stored in the storage to the apparatus. Further, the cardiography equipment 10 includes a display 11 which displays data. The cardiography equipment 10 can display an operation state of the cardiography equipment 10 at the display 11 during operation. The subject can grasp whether or not the cardiography equipment 10 normally operates, that is, whether or not measurement is normally performed by confirming the display 11 during the measurement period.

At the torso portion of the front body 31, a hook and loop fastener B side (loop side) 40 is sewed so that a flat rubber embedded in the rear body 32 can be fixed with an equal extension degree in accordance with the size of the under bust of the wearer. Further, side tabs 36 are attached at both ends of the torso portion of the rear body 32. The front body 31 is connected to the rear body 32 at both sides (side portions) by a hook and loop fastener A side (hook side) provided on a rear side of the side tabs 36 being fixed with the hook and loop fastener B side 40 of the front body 31. When the hook and loop fastener A side is fixed, the side tabs 36 are located around the under bust. The hook and loop fastener A side and the hook and loop fastener B side 40 function as a size adjustment mechanism that adjusts the pressure when the subject puts on the garment. While the example includes two right and left size adjustment mechanisms, the number of size adjustment mechanisms may be one or three or more.

A plurality of stitches 41 are provided on the hook and loop fastener B side 40 at intervals to make it easier to understand fastened positions of the side tabs 36. The stitches 41 correspond to scales indicating candidates for fastened positions of the side tabs 36 (adjustment positions for positioning). However, the side tabs 36 do not have to be fastened at the positions of scales and may be fastened at positions between scales. Further, one stitch 41 may be provided instead of a plurality of stitches 41 being provided. The stitches 41 are constituted with colored yarn different from color of the hook and loop fastener B side 40 as an example.

An interval of scales is equal to or greater than 1 cm and equal to or less than 8 cm at each of the right and left size adjustment mechanisms as an example. When there is one size adjustment mechanism, the interval of scales is equal to or greater than 2 cm and equal to or less than 16 cm as an example.

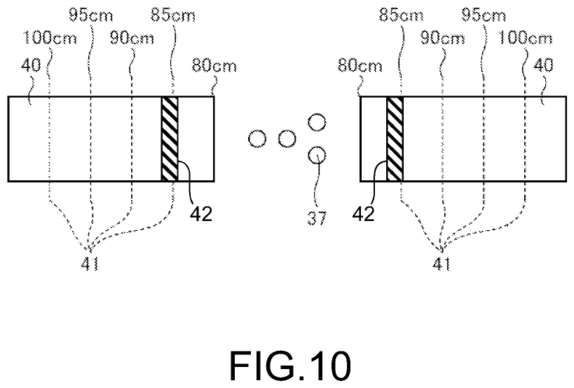

FIG. 4 is an enlarged view of the hook and loop fastener (B side) of the front body 31. FIG. 4 illustrates an example of a garment of M size which is to be applied to a size of the under bust of the subject from 80 cm to 100 cm. The subject fastens tips of the side tabs 36 at appropriate positions (fastened positions) in accordance with the size of the under bust. For example, when the size of the under bust of the subject is 90 cm, the subject fastens the tips of the right and left side tabs 36 to be respectively aligned with positions of the second stitches from the connector 37 of the cardiography equipment 10. Further, when the size of the under bust is 87 cm, the subject fastens the tips of the side tabs 36 to be aligned with positions moved from positions of the first stitches to the second stitches by 1 cm. In the example, the garment is provided to the subject in a state where the fastened positions are marked (see FIG. 10 which will be described later) to prevent the subject from fastening the tips of the side tabs 36 at wrong positions or to prevent the subject from forgetting the fastened positions.

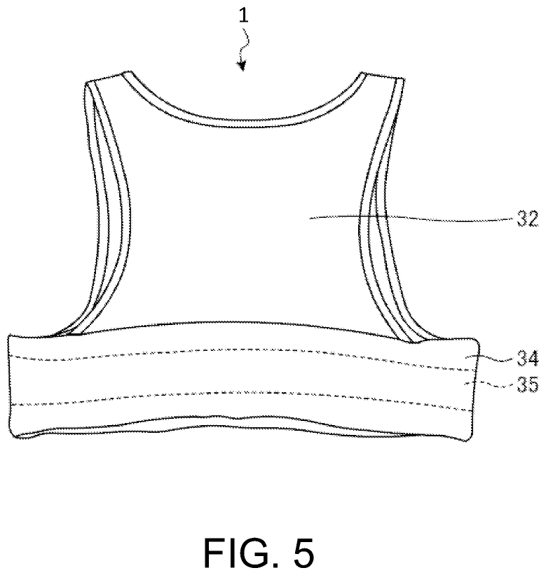

FIG. 5 is a rear view of the garment 1. A flat rubber is embedded into the torso portion 34 of the rear body 32 as an elastic body 35. As an example, by setting a length of the elastic body 35 at equal to or higher than 30% and equal to or less than 60% of the under bust of the subject, it is possible to acquire biological signals while the electrodes 20, 21 and 22 supported at the garment body portion 30 make contact with the skin of the subject with an appropriate pressure, that is, without the subject feeling strong compression by putting on the garment. Further, the elastic body 35 is approximately from 25 mm to 50 mm wide as an example. The elastic body 35 requires force equal to or greater than 3N and equal to or less than 9N to extend in a long axis direction by 30% as an example. Further, the elastic body 35 requires force equal to or greater than 2N and equal to or less than 6N to extend in the long axis direction by 20% as an example.

While FIG. 4 illustrates an example of the garment of the M size which is to be applied to the size of the under bust of 80 cm to 100 cm, a plurality of sizes are prepared in accordance with ranges of the size of the under bust. As an example, an S size is prepared for the size of the under bust of 60 cm to 80 cm, and an L size is prepared for 100 cm to 120 cm. A size of the garment body portion (a length (height) in a longitudinal direction and a length (width) in a lateral direction on paper in FIG. 2) is different depending on a difference in a size of the garment. Further, in each size, the hook and loop fastener (B side) and respective stitches on the hook and loop fastener (B side) are arranged to be able to fix the side tabs at appropriate positions in accordance with the size of the under bust of the wearer. Numerical values described here are merely an example. For example, the size of the garment may be prepared for each size range such as 10 cm in place of 20 cm. Further, the ranges of the under bust may overlap between sizes. For example, the M size may be prepared for 80 cm to 100 cm, and the L size may be prepared for 95 cm to 115 cm. Further, other sizes such as an SS size or an LL size may be provided. Still further, only two sizes may be provided for the garment.

The doctor, the laboratory technician or other health personnel orders a garment to the medical equipment sales agency with which they have partnership in advance using the information processing apparatus 100. Hereinafter, a person who operates the information processing apparatus 100 will be referred to as an operator. The operator inputs information necessary to order the garment to the information processing apparatus 100 and transmits order information on the garment to the order reception apparatus 200 of the medical equipment sales agency using the information processing apparatus 100. When a plurality of doctors and laboratory technicians and the like operate the information processing apparatus 100, the information processing apparatuses operated by the respective persons may be the same apparatus or may be different apparatuses having similar functions.

As illustrated in FIG. 1, the information processing apparatus 100 includes an input device 101, a display 102, a storage 103, a communication device 104 and an order generator 105. A hardware configuration of the information processing apparatus 100 is similar to that of a typical computer including a processor such as a CPU, a storage apparatus, a display apparatus, an input apparatus, a communication interface and the like.

The input device 101 is an apparatus for inputting data, and, for example, is a mouse, a keyboard, a touch panel or the like. The input device 101 may be a speech input device such as a microphone which performs input using speech. Examples of data to be input can include instruction information by the operator, information on the subject or the like.

The display 102 is a display apparatus that displays data and is, for example, a liquid crystal display apparatus, an organic EL display apparatus, a CRT display apparatus or the like.

The storage 103 is an apparatus that stores data. The storage 103 stores data input from the input device 101. Further, the storage 103 stores various kinds of application (such as an operating system (OS) and order application) necessary to implement operation of the example. Further, the storage 103 may store information on the subject as an electronic medical record. Further, the storage 103 may store data generated by the order generator 105 which will be described later, and data received at the communication device 104. Specific examples of the storage 103 can include a memory apparatus, a hard disk, an SSD or the like. The storage 103 may be connected from outside to be detachable via a connection interface (such as a USB port and a memory slot) provided at the information processing apparatus 100.

The communication device 104 is connected to the communication network 700 by wire or wirelessly and performs communication with other apparatuses in accordance with communication protocol. Specific examples of other apparatuses can include the order reception apparatus 200, the data processing apparatus 300, the analysis apparatus 500 and the communication terminal 600.

The order generator 105 generates an order input screen for ordering a garment and causes the order input screen to be displayed at the display 102. The order generator 105 is implemented by a processor such as a CPU being caused to execute the order application. The operator inputs information necessary to order on the order input screen via the input device 101.

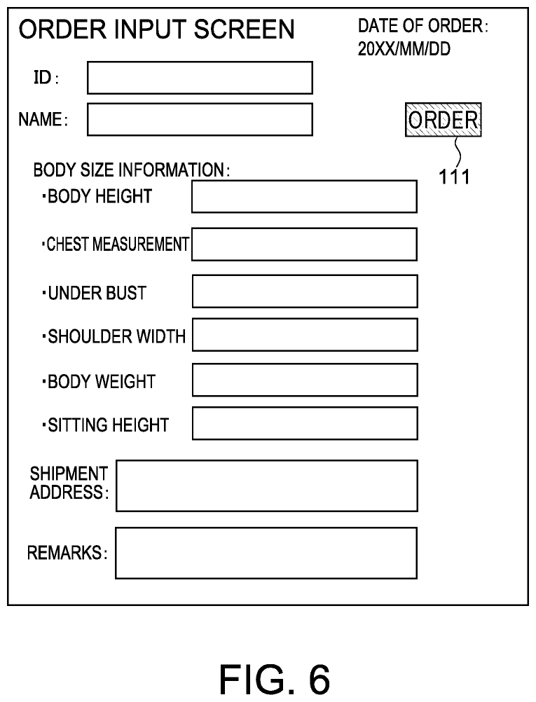

FIG. 6 illustrates an example of the order input screen. The order input screen includes a plurality of input items including an ID of the subject, name of the subject, size information on the subject, shipping address of the garment (for example, a dwelling place or address of the subject) and remarks. Today's date is displayed as date of order at an upper right portion of the screen. Arbitrary information is input in the remarks. For example, date and time at which the subject desires to receive the garment may be designated and input. Information (such as name) of the medical equipment sales agency may be displayed on this order input screen. Further, email address information on the order reception apparatus 200 may be displayed. A transmission instruction is provided to the order application by the operator entering data at respective input items and clicking an order button 111.

The order application generates order information on the garment upon receipt of the transmission instruction from the operator. The order information includes various kinds of information input on the order input screen as an example. The order generator 105 outputs the generated order information to the communication device 104. The communication device 104 transmits the order information to the order reception apparatus 200 via the communication network 700. The order information is transmitted by email as an example. In this example, email address information on the order reception apparatus 200 which is a transmission destination of the order information may be set in advance in the order application or may be input by the operator on the order input screen.

A transmission method of the order information is not limited to email. For example, a web server that receives orders may be provided on the order reception apparatus 200 side, and the order generator 105 may access an URL of the web server. In this example, the order information is transmitted to the order reception apparatus 200 by the operator inputting information necessary to order from a screen which is provided by the web server and displayed at the display 102 of the information processing apparatus 100. Alternatively, the order generator 105 may transmit the order information via fax machine connected to the information processing apparatus 100 via a network. In this example, the fax machine may transmit the order information in a form of an electronic file to the order reception apparatus 200. Alternatively, the order generator 105 may transmit the order information to a printer connected to the information processing apparatus 100 via a network and may cause the printer to print the order information. In this example, the operator may send a sheet which describes the order information to the medical equipment sales agency by mail or by courier or by fax.

A function of inputting an order on a screen of an electronic medical record of the subject may be provided. In this example, the screen of the electronic medical record can be used as the order input screen. In this event, a function of inputting an order instruction such as an order button or order menu is added on the screen of the electronic medical record. When the electronic medical record does not include part of items required to order the garment, it is only necessary to add the items to the electronic medical record.

The medical equipment sales agency is a medical equipment sales company. The medical equipment sales company accepts an order of the garment from the medical institution (information processing apparatus 100). The medical equipment sales company determines a size of the garment to be provided to the subject and fastened positions of the side tabs using the order reception apparatus 200. The medical equipment sales agency prepares the garment of the determined size, marks the determined fastened positions of the side tabs and sends the garment to the subject.

In more detail, the order reception apparatus 200 includes an input device 201, a display 202, a storage 203, a communication device 204, a garment selector 205, and a shipment instruction device 206. A hardware configuration of the order reception apparatus 200 is similar to that of a typical computer including a processor such as a CPU, a storage apparatus, a display apparatus, an input apparatus, a communication interface and the like. The order reception apparatus 200 is operated by staff of the medical equipment sales agency.

The input device 201, the display 202, the storage 203, and the communication device 204 have functions similar to those of the input device, the display, the storage and the communication device of the information processing apparatus 100 except content or a type of data or application to be dealt with.

The communication device 204 passes the order information received from the information processing apparatus 100 to order reception application. The order reception application holds the order information that has received so far. The order reception application displays a list of the order information. The staff selects unprocessed order information and displays content of the selected order information on a screen. The staff activates garment selection application to determine the size of the garment and the fastened positions of the side tabs appropriate for the subject. The garment selection application is executed by a processor such as a CPU. By this means, functions of the garment selector 205 are implemented. The garment selector 205 displays a screen of the garment selection application (garment selection screen) at the display 202.

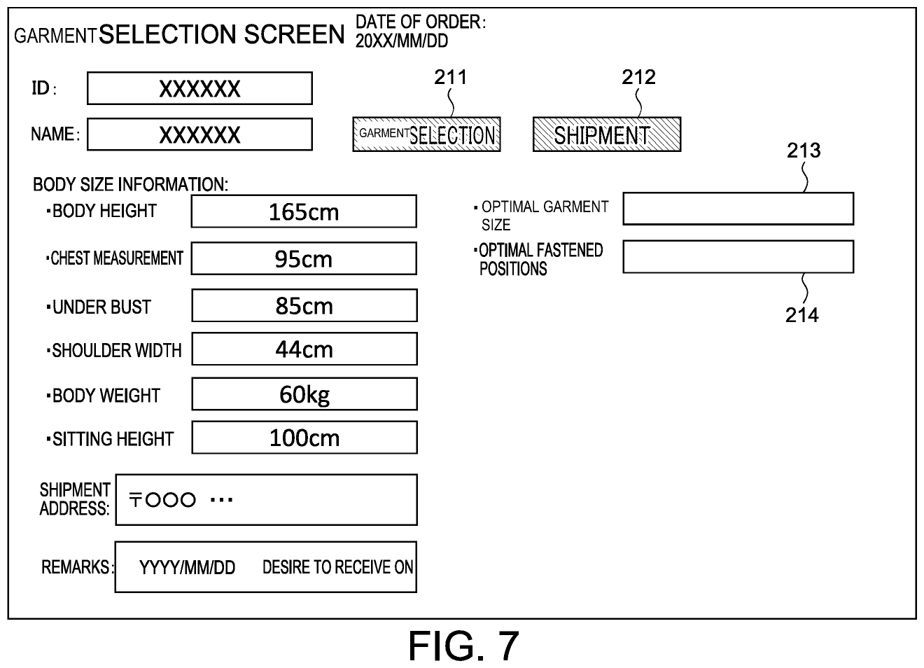

FIG. 7 illustrates an example of the garment selection screen. Content of the selected order information is displayed on the garment selection screen. Further, a garment selection button 211 to be used by the staff to give an instruction of garment selection and calculation of the fastened positions and a shipment button 212 for shipment of the garment are disposed. Further, the garment selection screen includes a field 213 that stores information on an optimal garment size calculated for the subject, and a field 214 that stores information on optimal fastened positions determined for the subject. At this point, no value is stored in the fields 213 and 214.

When the staff clicks the garment selection button 211, a garment selection instruction is provided to the garment selection application. The garment selection application selects a size of the garment optimal for the subject and calculates optimal fastened positions of the side tabs on the basis of the size information on the subject.

In the example, the size of the garment and the fastened positions are determined on the basis of the under bust size of the subject. As an example, the garment size includes S, M and L. A plurality of fastened positions are set in accordance with each size. For example, in an example of the size M illustrated in FIG. 4, the fastened positions are set at 1 cm intervals for the under bust from 80 cm to 100 cm. A setting example of the fastened positions will be described below. P_{M}1, P_{M}2, . . . , P_{M}21 are identifiers of the respective fastened positions.

80 cm: position of the first stitch (P_{M}1)

81 cm: position moved from the position of the first stitch to the second stitch by 5 mm (P_{M}2)

82 cm: position moved from the position of the first stitch to the second stitch by 1 cm (P_{M}3)

83 cm: position moved from the position of the first stitch to the second stitch by 1.5 cm (P_{M}4)

84 cm: position moved from the position of the first stitch to the second stitch by 2 cm (P_{M}5)

85 cm: position of the second stitch (P_{M}6)

86 cm: position moved from the position of the second stitch to the third stitch by 5 mm (P_{M}7)

87 cm: position moved from the position of the second stitch to the third stitch by 1 cm (P_{M}8)

88 cm: position moved from the position of the second stitch to the third stitch by 1.5 cm (P_{M}9)

99 cm: position moved from a position of the third stitch to the fourth stitch by 2 cm (P_{M}20)

100 cm: position of the fourth stitch (P_{M}21)

Also, for size S, a plurality of fastened positions P_{S}1, P_{S}2, P_{S}21 are set at 1 cm intervals in a range of the under bust from 60 cm to 80 cm in a similar manner. Also, for size L, a plurality of fastened positions P_{L}1, P_{L}2, P_{L}21 are set at 1 cm intervals in a range of the under bust from 100 cm to 120 cm. While an example where the fastened positions are set at 1 cm intervals has been described here, the interval may be 0.5 cm, or may be other values. It is only necessary to determine the interval in accordance with granularity of a measurement size of the under bust.

The garment selection application holds correspondence information in which sizes of the garment are associated with the fastened positions of the side tabs for each under bust size as an example. The correspondence information may have a table form or may be embedded in the garment selection application as a program code.

The garment selection application determines the size of the garment and the fastened positions of the side tabs that are optimal for the subject on the basis of the under bust size of the subject and the above-described correspondence information. For example, when the under bust size of the subject is 85 cm, the garment selection application determines that the garment size optimal for the subject is M, and the fastened positions of the side tabs optimal for the subject are P_{M}6. The garment selection application stores values indicating the determined garment size and the determined fastened positions of the side tabs in the fields 213 and 214 on the garment selection screen.

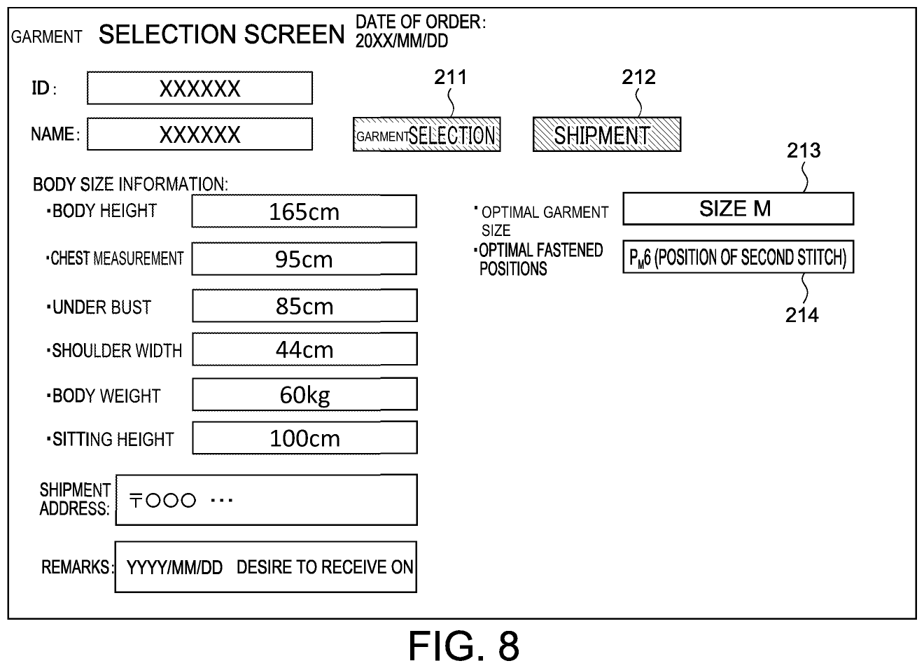

FIG. 8 illustrates an example where the values indicating the size of the garment and the fastened positions determined by the garment selection application are stored in the fields 213 and 214 on the garment selection screen. The garment size M and the fastened positions P_{M}6 of the side tabs are determined for the subject.

While the size of the garment and the fastened positions of the side tabs have been determined on the basis of the under bust size here, a determination method is not limited to this. For example, the size of the garment and the fastened positions of the side tabs may be determined further using at least one of a body height, a chest measurement, a sitting height and a body weight of the subject. For example, the size of the garment and the fastened positions of the side tabs may be calculated on the basis of the under bust and the chest measurement. As described above, the size is not limited to S, M and L, and more or less sizes may be provided.

Further, the size of the garment and the fastened positions of the side tabs may be determined by the staff. In this example, the staff determines the size of the garment and the fastened positions of the side tabs from the above-described correspondence information on the basis of the under bust size of the subject. The staff inputs values of the determined garment size and fastened positions in the fields 213 and 214.

After the values of the garment size and the fastened positions are stored in the fields 213 and 214, a shipment screen to perform a shipment procedure is displayed by the staff clicking the shipment button 212.

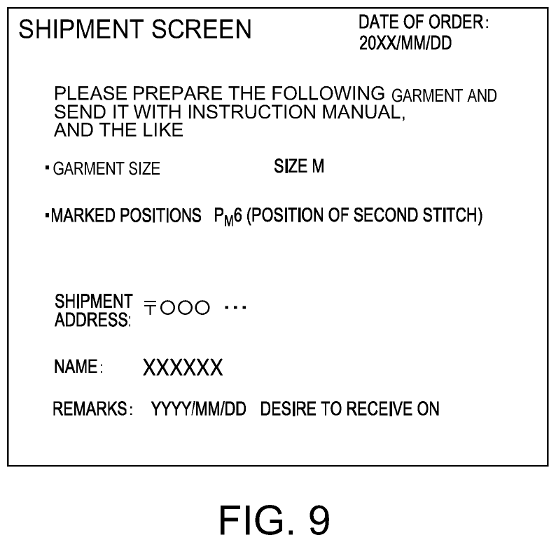

FIG. 9 illustrates an example of the shipment screen. The staff prepares the garment and marks the fastened positions with the content displayed on the shipment screen and sends an electrocardiogram measurement set including the garment on which the marks are provided, an instruction manual and the like to the subject, specifically, as follows.

After the staff confirms the garment size and the fastened positions of the side tabs optimal for the subject, the staff operates the inventory management apparatus 400 to confirm whether or not the garment of the size is left in stock. The inventory management apparatus 400 is an apparatus for managing inventory of the garments stored for each size within a warehouse 401. The inventory management apparatus 400 is a computer including an input apparatus, a display apparatus, a processor such as a CPU and a communication apparatus. The staff picks up the garment of the size of the subject from the warehouse 401 after confirming whether or not the garment is left in stock and marks the fastened positions for the subject on the garment. The fastened positions may be marked with a felt pen, with yarn of specific color, with an arrow mark or using other methods.

FIG. 10 illustrates an example where the positions of the second stitches (P_{M}6) on the garment of the size M illustrated in FIG. 4 are marked. Marks 42 are provided at the second stitches.

The fastened positions may be marked with machine prepared to mark as well as being marked by the staff. For example, data indicating the fastened positions are input to the machine, and the garment on which the marks are to be provided is placed at the machine. The machine automatically provides marks on the garment by the staff giving a marking instruction to the machine. The data indicating the fastened positions may be automatically input to the machine by the machine being made to coordinate with the order reception apparatus 200 or the inventory management apparatus 400.

The garments on which the respective fastened positions (P_{S} 1 to P_{S}21 in an S size, P_{M}1 to P_{M}21 in an M size, and P_{L}to P_{L}21 in an L size) are marked may be prepared in advance and stored in the warehouse 401. In this example, the staff only requires to pick up the garment of the size of the subject, on which the fastened positions determined for the subject are marked from the warehouse 401.

While the order reception apparatus 200 and the inventory management apparatus 400 are separately provided in FIG. 1, the order reception apparatus 200 and the inventory management apparatus 400 may be integrated by the functions of the inventory management apparatus 400 being added to the order reception apparatus 200. Further, the inventory management apparatus 400 does not have to be provided.

The staff packages the electrocardiogram measurement set including the garment on which the marks are provided, the instruction manual and the like, and sends the set to the subject at shipment address of the subject. The set may be sent by courier or by mail or other ways. An invoice to be pasted on the package may be automatically printed by the shipment application, the garment selection application or the like, outputting shipment information on the subject to a printer at a predetermined timing (for example, at a timing at which a shipment instruction is given) or may be created through operation by the operator.

While the staff picks up the garment from the warehouse and packages and sends the garment here, all or part of these series of operation can be automatically performed by utilizing a drone or an autonomous driving car.

For example, an automated conveyance apparatus (which may be a mobile robot, a belt conveyer or other types of apparatuses) that coordinates with the order reception apparatus 200 or the inventory management apparatus 400 may be provided in the warehouse 401 and may be caused to convey garments in the warehouse 401. For example, when the shipment button 212 is clicked on the garment selection screen, a conveyance instruction is transmitted from the order reception apparatus 200 to the automated conveyance apparatus directly or via the inventory management apparatus 400 at the same time or before or after the shipment screen is displayed. The automated conveyance apparatus automatically picks up the garment of the size designated in the conveyance instruction from a shelf on which garments are stored for each size and places the garment to a predetermined location. The staff picks up the garment from the predetermined location.

Further, when garments with marks are stored, the automated conveyance apparatus automatically passes the conveyed garment to an automated packaging apparatus. The automated packaging apparatus packages the garment and creates and pastes an invoice. The automated packaging apparatus passes the package to an automated transfer apparatus (such as a drone and an autonomous driving car) and the automated transfer apparatus automatically sends the package to the shipment address of the subject. The shipment address may be transmitted from the inventory management apparatus 400 or the order reception apparatus 200 to the automated transfer apparatus or may be read by the automated transfer apparatus from the invoice. There are various possible methods for sending the garment to the subject after the garment size and the fastened positions optimal for the subject are determined in this manner, and any method may be used.

The subject receives the electrocardiogram measurement set sent from the medical equipment sales agency. The subject puts on the garment in accordance with the instruction manual included in the package and explanation received from the health personnel (such as the doctor and the laboratory technician) of the medical institution in advance and records an electrocardiogram. For example, the subject puts on the garment directly on the skin of the upper body and aligns the tips of the side tabs with the marked positions to thereby fix the garment on the body.

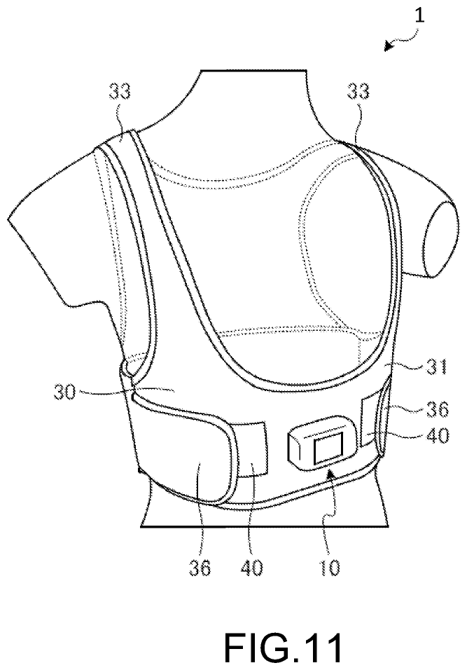

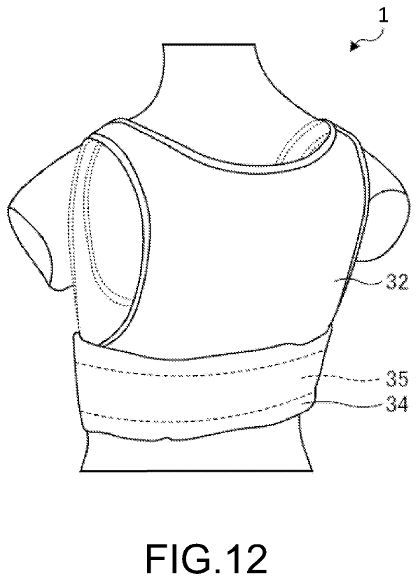

FIG. 11 is a view of the subject seen from a diagonally forward right direction when the subject puts on the garment 1. FIG. 12 is a rear view of the subject seen from a diagonally backward left direction when the subject puts on the garment 1. Misalignment of the garment body portion 30 is prevented because the front body 31 is connected to the rear body 32 with the shoulder belt 33. The garment of the size appropriate for the subject is provided, and the subject fixes the tips of the side tabs at appropriate fastened positions so that the electrodes 20, 21 and 22 (FIG. 3) make contact with the skin of the subject with an appropriate pressure. The subject therefore does not feel strong compression by putting on the garment. This enables the subject to record an electrocardiogram in a comfortable environment and enables biological signals of the subject to be acquired at an appropriate level that allows correct electrocardiographic analysis. Further, marking of the fastened positions allows the subject to easily understand the positions where the side tabs 36 are to be fastened.

The subject activates the cardiography equipment by powering on the cardiography equipment before or after putting on the garment. By this means, measurement is started. The measurement is performed for a measurement period (for example, one week) determined on the basis of the diagnosis by the doctor. The subject keeps the garment on and intermittently performs measurement except a limited period while the subject changes his/her clothes, takes a bath or the like. When the cardiography equipment and the electrodes are detachable from the garment, the subject may detach the cardiography equipment and the electrodes from the garment and may wash or clean a garment portion.

The subject has the communication terminal 600. The communication terminal 600 is a terminal such as a smartphone, a mobile phone, a tablet terminal, a notebook personal computer (PC) and a desktop PC. The subject may contact the health personnel (such as the doctor and the laboratory technician) of the medical institution or the staff of the medical equipment sales agency using the communication terminal 600 by email, by phone or the like.

The subject returns the garment which the subject has put on to the medical equipment sales agency after the measurement period. The garment may be returned using a method designated by the medical equipment sales agency in advance such as by mail or by courier. In a similar manner to when the garment is sent to the subject, the garment may be returned using a drone, an autonomous driving car or the like. The cardiography equipment may have a built-in timer, sound source and speaker, and may notify end of the measurement period to the subject with the timer. In place of or in addition to the sound source and the speaker, the cardiography equipment may include a vibrator and may notify end of the measurement period to the subject by driving the vibrator. The subject makes settings of the timer upon start of measurement as an example. Further, information on the measurement period may be notified from the information processing apparatus 100 to the order reception apparatus 200, and instructions which describe the measurement period may be included in the electrocardiogram measurement set sent to the subject. When a duration of the measurement period is determined in advance, operation of notifying the information on the measurement period from the information processing apparatus 100 to the order reception apparatus 200 is not required.

The data processing apparatus 300 of the medical equipment sales agency includes an input device 301, a display 302, a storage 303, a communication device 304, a reader 305, and an analysis request device 306. A hardware configuration of the data processing apparatus 300 is similar to that of a typical computer including a processor such as a CPU, a storage apparatus, a display apparatus, an input apparatus, a communication interface and the like. The data processing apparatus 300 may be the same hardware apparatus as the order reception apparatus 200 or the inventory management apparatus 400 or may be a hardware apparatus different from the order reception apparatus 200 or the inventory management apparatus 400. The data processing apparatus 300 is operated by the staff of the medical equipment sales agency.

The input device 301, the display 302, the storage 303, and the communication device 304 have functions similar to those of the input device, the display, the storage and the communication device of the information processing apparatus 100 or the order reception apparatus 200 except content or a type of data or application to be dealt with.

The staff connects the cardiography equipment loaded on the garment returned from the subject to the reader 305 by wire or wirelessly and causes the reader 305 to read out measurement data (electrocardiogram data) stored in the cardiography equipment. The reader 305 writes the read measurement data in the storage 303 as an electrocardiographic file. Further, the reader 305 may write the read data in a computer readable recording medium. Examples of the recording medium can include a CD-R, a USB memory, an SSD, a memory card and the like.

There are various configurations where the reader 305 reads out measurement data from the cardiography equipment. For example, the measurement data may be read out from the cardiography equipment through wireless local area network (LAN) communication, Bluetooth, near field communication or the like. Further, the reader 305 may be connected to the cardiography equipment by wire using a serial cable or the like, and the measurement data may be transmitted from the cardiography equipment to the reader 305 through serial transfer. Further, a dedicated data transfer scheme may be defined, and the measurement data may be transferred in accordance with this scheme. The measurement data may be transferred using a method other than the methods described here.

The analysis request device 306 is implemented by a processor such as a CPU being caused to execute analysis request application. The staff selects the measurement data (electrocardiographic file) for which an analysis request is to be made and gives an instruction to generate and transmit analysis request information via a user interface screen provided by the analysis request application.

The analysis request device 306 generates analysis request information to request analysis of the electrocardiogram for the measurement data selected by the staff. The analysis request information includes the measurement data for which an analysis request is to be made, information on the subject and the like. The information on the subject may include name and an ID of the subject or may include only an ID. The analysis request device 306 outputs the generated analysis request information to the communication device 304.

The communication device 304 transmits the received analysis request information to an analysis apparatus 500 of the electrocardiographic analysis institution via the communication network 700. Any method may be used to transmit the analysis request information to the analysis apparatus 500. The analysis request information may be transmitted by e-mail or may be transmitted using a file exchange system or may be uploaded to a dedicated web server prepared at the electrocardiographic analysis institution. Further, the analysis request information to be transmitted may be encrypted.

While the analysis request information is transmitted via the communication network 700 here, a recording medium in which the measurement data is stored and an analysis request form may be transmitted to the electrocardiographic analysis institution by mail, by courier or the like. Further, the measurement data and the analysis request form may be transmitted to the electrocardiographic analysis institution by fax. The measurement data and the analysis request form may be provided to the electrocardiographic analysis institution using other methods.

The analysis apparatus 500 provided at the electrocardiographic analysis institution receives the analysis request information from the data processing apparatus 300. A hardware configuration of the analysis apparatus 500 is similar to that of a typical computer including a processor such as CPU, a storage apparatus, a display apparatus, an input apparatus, a communication interface and the like. The analysis apparatus 500 is operated by a staff of the electrocardiographic analysis institution.

The staff operates the analysis apparatus 500 to display the analysis request information received from the data processing apparatus 300 on a screen. The analysis apparatus 500 is equipped with electrocardiographic analysis application. The staff activates the electrocardiographic analysis application, causes the electrocardiographic analysis application to load the measurement data included in the analysis request information and execute analysis processing. The analysis apparatus 500 displays an analysis result on a screen.

The analysis apparatus 500 creates an analysis report on the basis of the analysis result. The analysis report includes information on the measurement period of the subject, information on the subject, the analysis result of the electrocardiogram and the like, as an example.

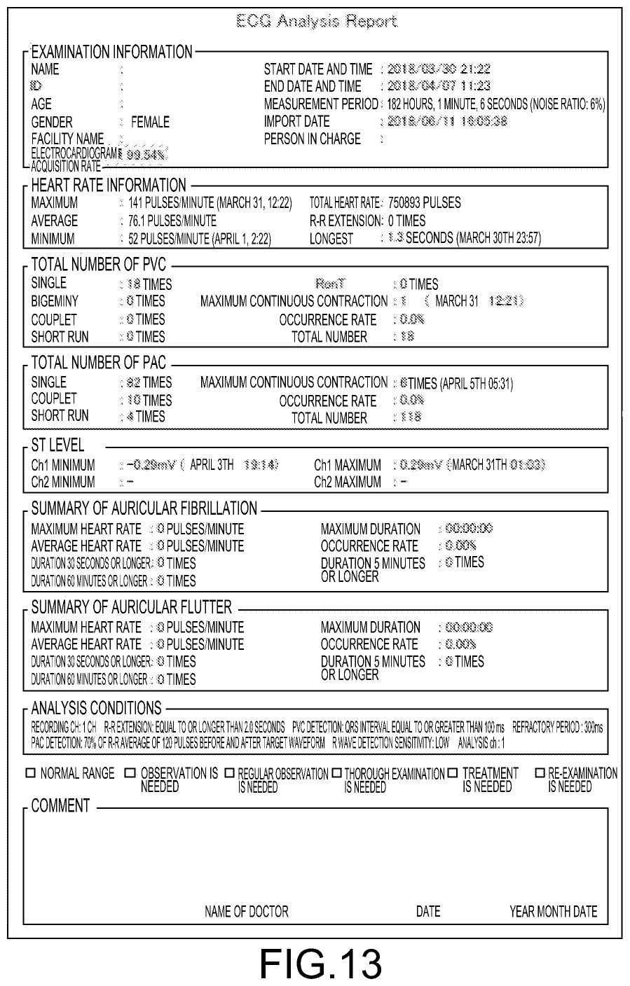

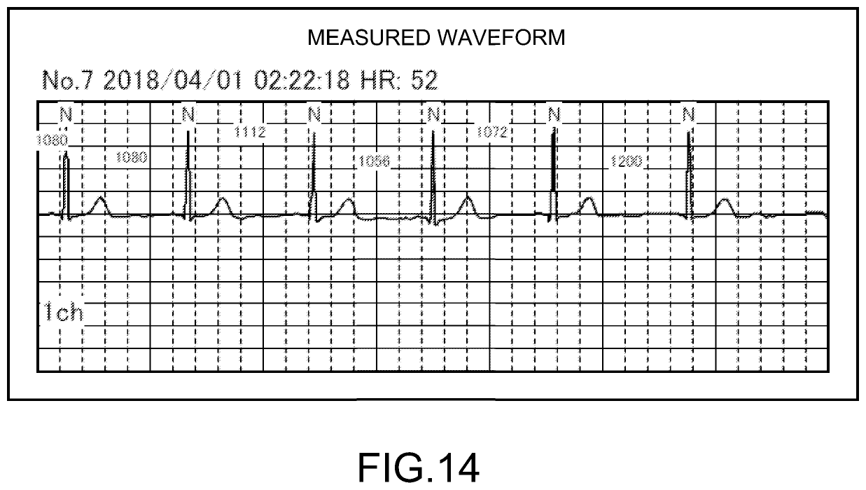

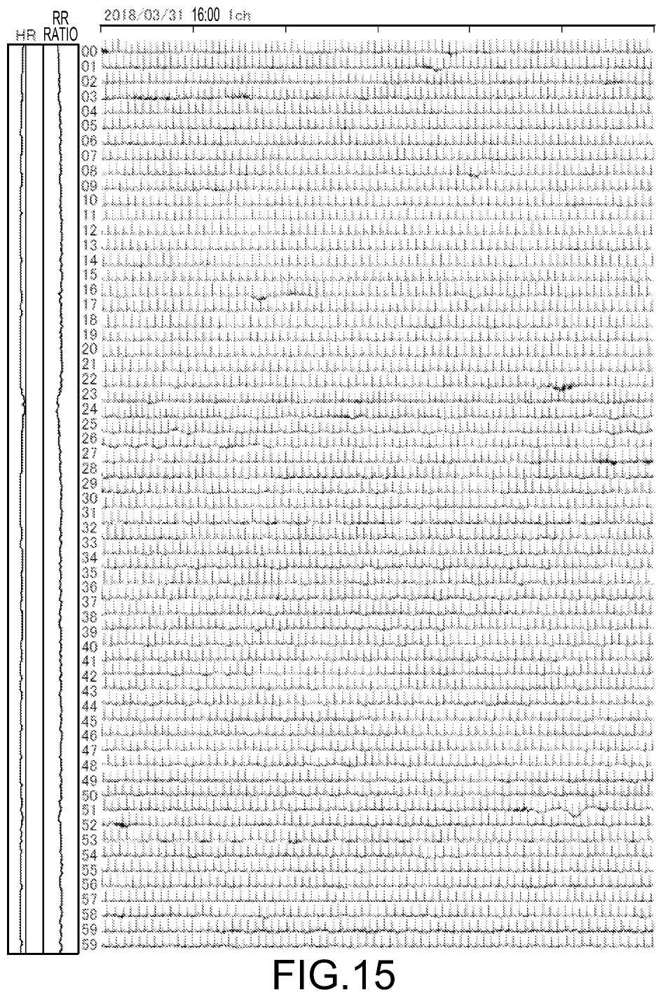

FIGS. 13 to 15 illustrate part of the analysis report. FIG. 13 illustrates a front cover portion of the analysis report in which overview of the analysis result is summarized. Measurement results of heart rate information, premature ventricular contraction (PVC), premature atrial contraction (PAC), an ST level, auricular fibrillation and auricular flutter are summarized in one page. A stable electrocardiogram that can be analyzed is obtained during a long period. FIG. 14 illustrates one of measured waveforms recorded in the analysis report. FIG. 15 illustrates compressed waveforms which indicate the electrocardiogram of one hour summarized on one page.

The staff operates the analysis apparatus 500 to transmit data of the analysis report to the information processing apparatus 100. The data of the analysis report may be transmitted by email, may be transmitted using a file exchange system or may be uploaded to a dedicated web server of the medical institution. The data of the analysis report to be transmitted may be encrypted.

The doctor displays the received analysis report on a screen and diagnoses a state of the subject on the basis of the electrocardiogram and the analysis result. The doctor enters the result of the diagnosis in, for example, the electronic medical record. The doctor interviews the subject upon next visit to the hospital and tells the diagnosis result. When the subject has a heart disease (abnormal cardiac rhythm) as a result of the diagnosis, the doctor discusses and determines courses of treatment in the future with the subject.

While the data of the analysis report is transmitted to the information processing apparatus 100 via the communication network 700 here, a recording medium including the data of the analysis report may be sent to the medical institution by mail, by courier or the like. Alternatively, the data of the analysis report may be transmitted by fax. The analysis report may be provided using other methods.

Further, the electrocardiographic analysis institution may transmit the data of the analysis report to the medical institution via the medical equipment sales agency. In this example, the communication device 304 of the data processing apparatus 300 receives the data of the analysis report from the analysis apparatus 500 and stores the received data in the storage 303. The staff operates the data processing apparatus 300 to transmit the data of the analysis report to the information processing apparatus 100. The communication device 104 of the information processing apparatus 100 receives the data of the analysis report transmitted from the data processing apparatus 300 and stores the received data in the storage 103. The doctor displays the received analysis report and diagnoses a state of the subject on the basis of the electrocardiogram and the analysis result.

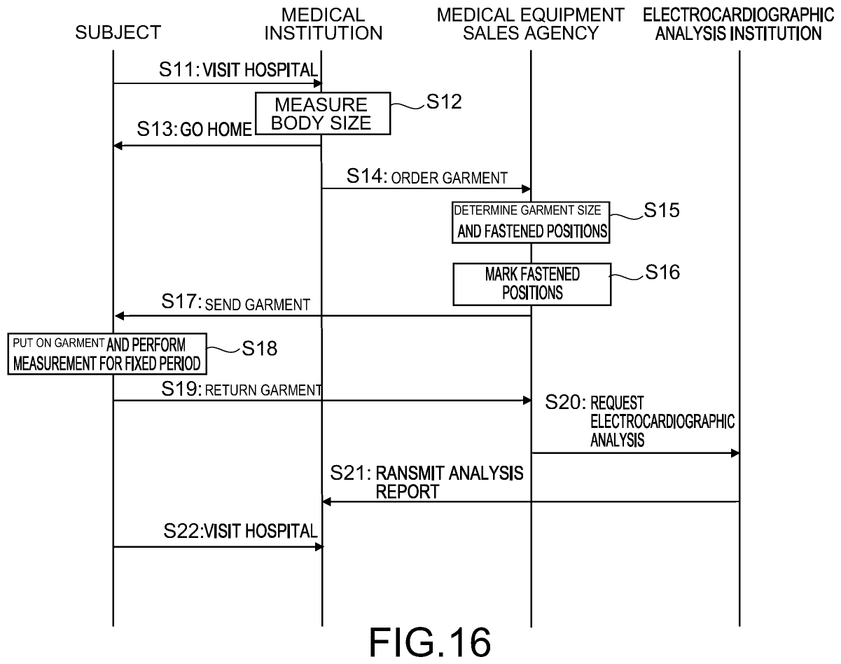

FIG. 16 is a sequence diagram of a specific example of the whole operation regarding the examination support system according to the example.

The subject visits the medical institution (S11). The doctor of the medical institution diagnoses the subject and enters the diagnosis result in the medical record. The medical record may be an electronic medical record or a paper medical record. The doctor judges that the subject is suspected to have a heart disease such as abnormal cardiac rhythm, determines to perform an electrocardiogram examination (Holter electrocardiogram examination) and issues a request form for Holter electrocardiogram examination. The request form may be issued using, for example, a request form print function provided at the electronic medical record. The doctor or the health personnel to whom the doctor provides directions gives an examination reception file including the request form to the subject. The subject presents the examination reception file to an examination room.

The laboratory technician measures the body size (such as under bust) of the subject and obtains size information including the under bust size (S12). Further, the laboratory technician explains how to use the garment which the subject puts on in the electrocardiogram examination and a measurement period. Then, the subject goes home (S13).

The doctor, the laboratory technician or other health personnel orders an electrocardiogram measurement set including the garment from the order screen using the information processing apparatus 100. Specifically, the doctor, the laboratory technician or other health personnel inputs the size of the under bust, name, an ID, shipment address or the like of the subject on the order screen. The information processing apparatus 100 transmits the order information on the electrocardiogram measurement set to the order reception apparatus 200 of the medical equipment sales agency (S14).

The order reception apparatus 200 receives the order information. The staff of the medical equipment sales agency activates the garment selection application of the order reception apparatus 200, inputs the size information (including at least the under bust) of the subject in the garment selection application and causes the garment selection application to calculate the garment size and the fastened positions (S15). The staff picks up the garment of the calculated size from the warehouse and marks the fastened positions (S16). When the marks are provided on the garment in advance, the staff only requires to pick up the garment on which the fastened positions are marked from the warehouse and does not have to do marking work. The staff sends the electrocardiogram measurement set including the garment on which the marks are provided, the instruction manual and the like, to the subject (S17). It is also possible to send the garment to the subject in a state where the fastened positions are not marked. In this example, the electrocardiogram measurement set may include a notification which notifies the fastened positions to the subject. Alternatively, data of the fastened positions may be transmitted from the order reception apparatus 200, the inventory management apparatus 400 or the like to the communication terminal 600 via the communication network 700.

The subject receives the electrocardiogram measurement set sent from the medical equipment sales agency. The subject puts on the garment in accordance with the instruction manual included in the electrocardiogram measurement set and explanation as to how to use the garment received from the health personnel (such as the doctor and the laboratory technician) in advance and records an electrocardiogram during the measurement period (S18). Specifically, the subject puts on the garment on the naked upper body and aligns the tips of the side tabs with the marked positions to thereby fix the garment on the body. The subject puts off the garment after the measurement period ends and returns the garment to the medical equipment sales agency (S19). It is also possible to return only the cardiography equipment loaded on the garment without returning the garment body portion or return the garment body portion at a later date.

The medical equipment sales agency receives the garment returned from the subject and causes the reader 305 of the data processing apparatus 300 to read out the measurement data (electrocardiographic data) stored in the cardiography equipment of the garment. The data processing apparatus 300 transmits analysis request information including the readout measurement data (electrocardiographic file) to the analysis apparatus 500 of the electrocardiographic analysis institution (S20).

The information processing apparatus 100 receives data of the analysis report including the analysis result from the analysis apparatus 500 (S21). The doctor of the medical institution diagnoses a state of the subject on the basis of the analysis result described in the analysis report. The doctor enters the diagnosis result in, for example, the electronic medical record. The doctor interviews the subject who visits the hospital (S22) and tells the diagnosis result. When it is recognized as a result of the diagnosis that the subject has a heart disease (abnormal cardiac rhythm), the doctor discusses and determines courses of treatment in the future with the subject.

The subject only requires to put on the garment sent from the medical equipment sales agency, record an electrocardiogram and return the garment which the subject has put on to the medical equipment sales agency after measurement so that it is possible to reduce the number of times the subject visits the medical institution. In known devices, for example, the subject needs to visit the hospital at least twice in addition to the first diagnosis and the diagnosis after the examination to have electrodes loaded on measurement positions of the body and the electrodes removed after 24 hours. In contrast, in this example, the subject only required to put on the sent garment and return the garment which the subject has put on and does not have to visit the hospital for loading and removal of electrodes so that it is possible to reduce the number of times of visiting the hospital. It is therefore possible to reduce load on the subject.

Further, the garment of the size appropriate for the subject is provided so that it is possible to stably dispose the electrodes at correct positions and perform measurement comfortably and easily while allowing the subject himself/herself to easily put on and off the garment. Further, the positions where the tips of the side tabs are to be fastened are marked on the garment so that the subject can fix the electrodes on the body with an appropriate pressure by fixing the tips of the side tabs at the marked positions. It is easy to always stably hold the electrode positions and the pressure so that it is possible to record an electrocardiogram for a long period beyond 24 hours comfortably and with high accuracy.

Further, it is possible to save work in loading the electrodes on the subject and technical guidance by the medical institution which has been required in the Holter electrocardiogram examination for 24 hours. It is therefore possible to reduce load on the medical institution. Further, the medical equipment is owned by the medical equipment sales agency and does not have to be owned by the medical institution so that it is possible to reduce load on the medical institution also in this viewpoint.

Further, the garment body portion can be cleaned and reused so that it is possible to reduce the number of garments in stock and expect reduction in overall medical expenses.

### Modified Example 1

In the above-described example, the subject returns the garment which the subject has put on to the medical equipment sales agency and the medical equipment sales agency reads out measurement data from the garment. In the modified example 1, the measurement data (electrocardiographic data) stored in a storage (such as a memory apparatus) inside the measurement equipment is transferred to the communication terminal 600 on the basis of operation by the subject, and the transferred measurement data is transmitted from the communication terminal 600 to the data processing apparatus 300. This enables the medical equipment sales agency to collect the measurement data early so that the medical equipment sales agency can make an analysis request to the electrocardiographic analysis institution early. This enables the doctor to obtain the analysis report earlier.

### Modified Example 2

While in the above-described example, the medical institution (information processing apparatus 100) transmits the order information to the medical equipment sales agency, the subject may transmit the order information using the communication terminal 600. In this example, the communication terminal 600 is equipped with application for creating and transmitting the order information, and the subject inputs size information such as the under bust size to the application.

### Modified Example 3

While in the above-described example, the garment selector 205 of the order reception apparatus 200 calculates the garment size and the fastened positions, when the medical institution keeps garments for trial fitting, at least one of the garment size and the fastened positions may be determined at the medical institution in step S12. In this example, a function for allowing input of at least one of the garment size and the fastened positions to the order input screen is provided at the order generator 105. The order generator 105 of the information processing apparatus 100 then calculates at least one of the garment size and the fastened positions from the under bust size, or the like. The information processing apparatus 100 transmits order information including the determined garment size and fastened positions to the order reception apparatus 200.

### Modified Example 4

While in the above-described example, the medical equipment sales agency sends the electrocardiogram measurement set to the subject, when the medical institution also plays a role as a garment sales agency, the medical institution may send the electrocardiogram measurement set. In this example, the medical institution determines the garment size and the fastened positions in accordance with modified example 3. Further, the subject may return the garment to the medical institution. Further, the medical institution may read out measurement data from the garment and may make an analysis request to the electrocardiographic analysis institution. Modified example 5

In the above-described example, a length adjustment mechanism may be provided at two shoulder belts 33 (see FIG. 3) or the torso portion 34 (see FIG. 5) so that the length of the two shoulder belts 33 or the torso portion 34 can be adjusted. The length adjustment mechanism may have any structure. For example, part of the shoulder belt may be formed in a circular shape and the length can be adjusted by changing a size of the circular portion. By adjusting the length in this manner, it is possible to prepare garments of a plurality of sizes such as S, M and L using garments which originally have the same size. Candidates for adjustment positions of the shoulder belt may be expressed with scales on the shoulder belt in a similar manner to the fastened positions of the side tabs. The garment may have a form without shoulder belts. Further, the order reception apparatus 200 or the information processing apparatus 100 may calculate the adjustment positions from the size information on the subject and provide a garment on which the determined positions are marked to the subject. It is only necessary to obtain and create correspondence relationship between the adjustment positions and the size information in advance from a number of samples.

The examples are not intended to limit the scope of this disclosure. These examples can be implemented in other various forms, and various omission, substitution and change can be made within the range not deviating from the scope of the appended claims. These examples and modified examples are incorporated into the scope of the disclosure and are also incorporated into the claims and ranges equivalent to those recited in the claims in a similar manner.

This application is based upon and claims the benefit of priority from the prior Japanese Patent Application No. 2018-225705, filed on Nov. 30, 2018, the entire contents of which are incorporated herein by reference.

1-15. (canceled)

16. An examination support method that supports an electrocardiogram examination of a subject using a garment on which medical equipment including cardiography equipment and a plurality of electrodes are loaded, the examination support method comprising:
a first apparatus selecting a garment to be put on the subject from the garments of a plurality of sizes on a basis of size information on the subject; and
a second apparatus connecting by wire or wirelessly with the medical equipment loaded on the garment that the subject has put on and reading out measurement data measured during a period in which the subject puts on the wear.

17. The examination support method according to claim 16, wherein the size information includes at least an under bust among the under bust, a body height, a chest measurement, a shoulder width, a sitting height, and a body weight.

18. The examination support method according to claim 16, wherein in the selecting, a size of the garment to be put on the subject is determined on a basis of the size information, and the garment having the determined size is selected.

19. The examination support method according to claim 16, wherein the garment includes one or a plurality of size adjustment mechanisms to adjust pressure on a torso portion of the subject.

20. The examination support method according to claim 19, wherein in the selecting, a size of the garment to be put on the subject and an adjustment position of the size adjustment mechanism are determined in accordance with the size information, and a garment with a mark indicating the determined adjustment position with which the size adjustment mechanism is to be aligned is selected among garments having the determined size.

21. The examination support method according to claim 29, wherein the garment has scales that align at a plurality of candidates for the adjustment position with which the size adjustment mechanism is to be aligned.

22. The examination support method according to claim 21, wherein a number of the size adjustment mechanisms is one, and an interval of the scales is equal to or greater than 2 cm and equal to or less than 16 cm, or the number of the size adjustment mechanisms is two, and an interval of the scales is equal to or greater than 1 cm and equal to or less than 8 cm.

23. The examination support method according to claim 20, further comprising:
transmitting information indicating the determined adjustment position to a communication terminal of the subject.

24. The examination support method according to claim 16, further comprising:
acquiring analysis result data by analyzing the measurement data read out by the second apparatus.

25. The examination support method according to claim 16, wherein the garment includes a shoulder belt.

26. The examination support method according to claim 16, wherein the cardiography equipment is able to operate for equal to or longer than 48 hours in a state where the cardiography equipment is fully charged.

27. The examination support method according to claim 16, wherein the electrodes and the cardiography equipment are detachable from the garment.

28. The examination support method according to claim 16,
wherein the cardiography equipment includes a display configured to display an operation state of the cardiography equipment, and
the examination support method comprises:
displaying the operation state of the cardiography equipment at the display.

29. The examination support method according to claim 16, comprising at least one of:
sending the selected garment to the subject; and
receiving the garment which the subject has put on and which is returned from the subject.

30. An examination support system that supports an electrocardiogram measurement of a subject using a garment on which medical equipment including cardiography equipment and a plurality of electrodes are loaded, the examination support system comprising:
an information processing apparatus that comprises:
an input device configured to input size information on the subject; and
a first communication device configured to transmit order instruction information on the garment on a basis of the size information;
an order reception apparatus that comprises:
a second communication device configured to receive the order instruction information from the information processing apparatus; and
a garment selector configured to select a garment to be put on the subject from the garments of a plurality of sizes in accordance with the order instruction information; and
a data processing apparatus that comprises:
a reader configured to connect by wire or wirelessly with the medical equipment loaded on the garment which the subject has put on and read out measurement data measured during a period in which the subject puts on the garment.
